(19) United States
(12) Patent Application Publication
(10) Pub. No.: US 2022/0363984 A1
(43) Pub. Date: Nov. 17, 2022

(54) PHOTO-RESPONSIVE COORDINATION COMPOUNDS WITH PHOTO-CONTROLLABLE ELECTRON-TRANSPORTING AND ELECTRICAL CONDUCTING PROPERTIES, AND FABRICATION OF ORGANIC ELECTRONICS AND ORGANIC RESISTIVE MEMORY DEVICES WITH PHOTO-SWITCHABLE PERFORMANCE

(71) Applicant: THE UNIVERSITY OF HONG KONG, Hong Kong (CN)

(72) Inventors: Wing-Wah, Vivian YAM, Hong Kong (CN); Cheok-Lam WONG, Hong Kong (CN); Yau-Hin HONG, Hong Kong (CN); Mei-Yee CHAN, Hong Kong (CN)

(21) Appl. No.: 17/753,849

(22) PCT Filed: Sep. 16, 2019
(86) PCT No.: PCT/CN2019/105938

§ 371 Date: Mar. 16, 2022

(51) Int. Cl. C09K 9/02; C07F 5/06; H01L 51/00

(52) U.S. Cl. CPC C09K 9/02; C07F 5/069; H01L 51/0081; C09K 2211/1037; C09K 2211/186; H01L 51/4206

(57) ABSTRACT

Disclosed is a new class of photo-responsive coordination compounds with at least one photochromic unit on a coordinating ligand. The photo-responsive coordination compounds are shown to be capable of acting as electroactive materials for the fabrication of organic memory devices as well as photo-controllable electron-transporting materials.

### TECHNICAL FIELD

Disclosed are photo-responsive coordination compounds with photo-controllable electron-transporting and electrical conducting properties that can be modulated by photo-irradiation. The photo-responsive coordination compounds can serve as a photoswitchable electron-transporting layer in organic electronics and as a photoswitchable electroactive layer in organic resistive memory devices.

### BACKGROUND

Organic electronics have been of particular interest in the past several decades because of their distinct and unique properties over the conventional inorganic counterparts in view of the great flexibility, simple manufacturing process, portable size and light-weight as well as the adaptive functionalities. Specifically, attempts have been made to incorporate the photo-responsive unit to the organic electronic devices so as to modulate the device performance by light. Particularly, a photo-responsive material will show intriguing changes in its optical, magnetic, mechanical, or electrical properties as a function of light irradiation. This represents an exciting area of study in material science, where researchers are venturing into a new world of materials with properties as yet unknown but that offer the promise of beneficial applications in health, industry, agriculture, and other fields. Considerable research has been dedicated to polymers with photo-responsive units embedded due to their versatility and relative ease of synthesis in combination with the spatial and temporal control provided by using light as trigger source.

Very recently, the incorporation of photochromic molecules into the organic framework has been documented as an effective means to achieve photo-controllable behaviors. Photochromic compounds possess at least two isomeric forms, which have different physical properties, including electronic properties, refractivities, and the like, and can be transformed from one form to another by photo-irradiation at prescribed wavelengths.

Among the photo-isomerizable species, diarylethene, spiropyran and spirooxazine which undergo the pericyclic reactions to yield photochromism have been of particular interests owing to their versatility in preparing photo-responsive functional molecules. In order to fulfill the practical use in device fabrication for optical recording and other optical functioning devices, both isomeric forms must be thermally stable and possess excellent durability for reversible photochromic reactivity. Notably, diarylethenes are superior to the other photochromic moieties owing to their excellent thermal stability, high fatigue resistance, prompt photo-responsiveness and high conversion ratio between open and closed conformers. Recently, it has been shown that the incorporation of nitrogen and sulfur-containing heterocycles into the “ethene” part of the diarylethene backbone, instead of derivatizing the pendants of the bis(thienyl)perfluorocyclopentene core, can enrich the photochromic and photophysical behaviors. It has also been demonstrated that superior bistability of both open and closed isomers can be achieved through the incorporation of phosphorus- and silicon-containing heterocycles by attaching phospholes and siloles to diarylethenes.

Meanwhile, research on metal-containing and coordination motif-containing diarylethenes has been an emerging field in the past decade and has been demonstrated to be a promising molecule scaffold for tuning the photochromism. The color of the photochromic complexes and coordination compounds can be effectively tuned through the coordination of the metal centers or main group atoms and, more importantly, the use of excitation light source with much lower energy to induce photo-isomerization is feasible through the readily accessible photo-sensitization. Furthermore, this can enhance the stability of the photoswitches. At the same time, by combining both the intrinsic properties of the coordination compound and the photo-responsiveness of the photochromic moiety, multi-functional smart materials can be readily obtained.

In view of the use of metal chelates and coordination derivatives in organic electronics nowadays, the integration of the photochromic unit to the coordination motif-containing scaffold is anticipated to provide a simple and efficient approach for modulating the performance of the photoswitches by simply applying the light irradiation without any need of the reconstruction of the entire molecular framework. This approach should be a great advantage for the rapid development in organic electronics, especially in organic resistive memory devices and photoswitches, such that the time for preparation and the cost of manufacture of new materials can be effectively reduced. Different from inorganic memories of which the performances are based on the amount of charges stored in the devices, the memory effect in organic resistive memory devices are strongly dependent on the electrical bistability of conductance (resistance), where a low-conductance (OFF) state will switch to a high-conductance (ON) state during operation. With the incorporation of the photochromic motif and coordination scaffold, it is anticipated that the variability and functionality of organic resistive memory devices can be enriched.

### SUMMARY

The following presents a simplified summary of the invention in order to provide a basic understanding of some aspects of the invention. This summary is not an extensive overview of the invention. It is intended to neither identify key or critical elements of the invention nor delineate the scope of the invention. Rather, the sole purpose of this summary is to present some concepts of the invention in a simplified form as a prelude to the more detailed description that is presented hereinafter.

As described herein, a novel class of photo-responsive coordination compounds with a photochromic moiety integrated into the coordinating ligand are generated. The photo-responsive coordination compounds can be utilized to serve as electroactive layer for the fabrication of organic resistive memory devices. Particularly, the photo-responsive coordination compounds exhibit high electron-transporting properties and high electrical conductivity upon light irradiation via photo-isomerization of the photochromic moiety. More importantly, the organic resistive memory devices based on the photo-switchable coordination compounds demonstrate binary memory behavior with high ON/OFF ratio of over 10⁴ and long retention time.

Furthermore, this new class of photo-responsive coordination compounds demonstrates photo-switchable electron-transporting properties and electrical conductivity under photo-irradiation. Such photo-controllable properties make this class of compounds promising electroactive materials for various organic electronics. Described below in one embodiment are the design, synthesis and studies of the photo-responsive coordination compounds bearing the photochromic moiety. Such compounds exhibit high electron-transporting properties and high electrical conductivity upon light irradiation.

Described in another embodiment are photo-switchable resistive memory devices based on the photo-responsive coordination compounds that are capable of reversibly undergoing photo-isomerization of the photochromic unit when photoirradiated with light. Such organic resistive memory devices demonstrate binary memory behavior with high ON/OFF ratio of over 10⁴ and long retention time. The photochromic unit can be, for example, diarylethene, spiropyran, spirooxazine or rhodamine. It is worth noting that under photo-irradiation, the electron-transporting and electrical conductivity, as indicated by the electron-transporting behavior and the memory effect of organic memory devices based on this class of compounds, can be modulated. The photo-responsive coordination compounds described herein provide a simple approach to obtain photo-responsive electron-transporting materials that can serve as active components in the fabrication of organic electronics and organic resistive memory devices. Photo-switchable memory performance can be readily achieved by photo-irradiation.

Also described herein is the preparation of photo-responsive coordination compounds having a chemical structure represented by the following general formula (I), which can be used as electroactive materials in organic electronic devices:

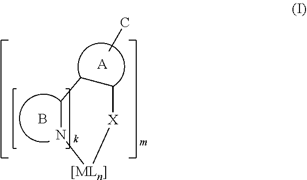

wherein

a) X can be oxygen, sulphur, selenium, NR or PR where R is alkyl, alkylaryl, cycloalkyl, alkoxy, benzyl, aryl, substituted aryl, heteroaryl, substituted heteroaryl or a heterocyclic group;

b) A is cyclic structure derivative of substituted or unsubstituted arene or heteroarene;

c) B, which can be fused with or singly-bonded to A, is cyclic structure derivative of substituted or unsubstituted heterocyclic group containing one or more nitrogen atoms;

d) C is a photochromic unit and preferably selected from, but not limited to, diarylethene, spiropyran, spirooxazine or rhodamine;

e) [MLₙ] represents the coordination unit containing a metal or main group element M and L is a ligand;

f) k is the number of rings in the cyclic structure derivatives and k is integer from 0 to 2;

g) n is the number of ligands and n is an integer from 0 to 4; and

h) m is the number of the photochromic ligand and m is an integer from 1 to 4.

It is found that the incorporation of the photochromic unit into the coordination compound can effectively modulate the electrical conductivity of the compounds by photo-irradiation and induces a photo-responsive electron-transporting property in such compounds. The compounds are thermally stable, highly soluble in most of the organic solvents and can readily form a thin film by either thermal deposition or spin-coating processes.

To the accomplishment of the foregoing and related ends, the invention comprises the features hereinafter fully described and particularly pointed out in the claims. The following description and the annexed drawings set forth in detail certain illustrative aspects and implementations of the invention. These are indicative, however, of but a few of the various ways in which the principles of the invention may be employed. Other objects, advantages and novel features of the invention will become apparent from the following detailed description of the invention when considered in conjunction with the drawings.

### BRIEF SUMMARY OF THE DRAWINGS

FIG. 1 shows a typical structure of an electron-only device for measuring the electrical conductivity of the photo-responsive coordination compounds.

FIG. 2 shows a schematic diagram of an organic resistive memory device.

FIG. 3 shows the UV-vis absorption spectral changes of compound 2 in degassed benzene upon excitation at 300 nm.

FIG. 4 shows the emission spectral changes of compound 2 in degassed benzene upon excitation at 337 nm.

FIG. 5 shows a plot of In(A/A₀) versus time for the absorbance decay of compound 2 at 543 nm at various temperatures in argon-flushed toluene solution; A denotes absorbance at time t and A₀ denotes the initial absorbance; solid lines represent the theoretical linear fits.

FIG. 6 shows the Arrhenius plot for the thermal backward reaction of the closed form of compound 2 in argon-flushed toluene solution.

FIG. 7 shows the UV-vis absorbance changes of compound 2 at 543 nm on alternate excitation at 300 and 525 nm over six cycles in degassed benzene solution at 298 K.

FIG. 8 shows the current density-voltage (J-V) curves of devices with active layer doped with compound 2 and their photo-responsive behavior.

FIG. 9 shows the J-V curves of devices with active layer doped with Alq₃ with or without exposure to photo-irradiation.

FIG. 10 shows the current-voltage (I-V) characteristics of an indium-tin-oxide (ITO)/active layer/AI device of compound 2 after light-irradiation.

FIG. 11 shows the stability of the photo-irradiated ITO/active layer/Al device of compound 2 in “OFF” and “ON” states under a constant stress (1.0 V).

FIG. 12 shows the scanning electron microscopy (SEM) image of the cross-section of the photo-irradiated device of compound 2.

### DETAILED DESCRIPTION

Described herein are photo-responsive coordination compounds that enable switching from a high resistive state (OFF state) to a low resistance state (ON state) upon exposure to light irradiation. The electron-transporting properties of the photo-responsive coordination compounds can be photo-modulated and at least 2-fold increase in electrical conductivity can be obtained under photo-irradiation. In addition, the photo-responsive coordination compounds simultaneously exhibit thermal stability and fatigue resistance.

Embodiments are directed to a new class of photo-responsive coordination compounds with photo-controllable electron-transporting and electrical conducting properties. The photochromic unit can be, for example, diarylethene, spiropyran, spirooxazine or rhodamine. The photo-responsive coordination compounds have the chemical structure shown in the generic formula (I):

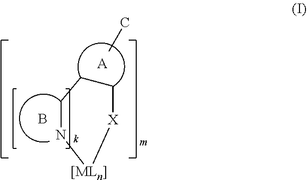

wherein

a) X can be oxygen, sulphur, selenium, NR or PR where R is alkyl, alkylaryl, cycloalkyl, alkoxy, benzyl, aryl, substituted aryl, heteroaryl, substituted heteroaryl or a heterocyclic group;

b) A is cyclic structure derivative of substituted or unsubstituted arene or heteroarene;

c) B, which can be fused with or singly-bonded to A, is cyclic structure derivative of substituted or unsubstituted heterocyclic group containing one or more nitrogen atoms;

d) C is a photochromic unit and preferably selected from, but not limited to, a diarylethene, a spiropyran, a spirooxazine or a rhodamine;

e) [MLₙ] represents the coordination unit containing a metal or main group element M and L is a ligand;

f) k is the number of rings in the cyclic structure derivatives and k is integer from 0 to 2;

g) n is the number of ligands and n is an integer from 0 to 4; and

h) m is the number of the photochromic ligand and m is an integer from 1 to 4.

Ring A is cyclic structure derivatives where the cyclic structures are independently selected from a 5- or 6-membered arene or heteroarene. The arene can be benzene, naphthalene, anthracene, pyrene, fluorene and derivatives thereof, and the heteroarene can be pyridine, pyrazole, imidazole, oxazole, isoxazole, thiazole, isothiazole, isoquinoline, pyrrole, pyrazine, pyridazine, pyrimidine, benzimidazole, benzothiazole, indole, triazole, tetrazole, pyran, oxadiazole, triazine, tetrazine, and derivatives thereof.

Ring B is cyclic structure derivative where the cyclic structure is independently selected from a 5- or 6-membered nitrogen-containing heteroarene or heterocycle known in the art. The heteroarene or heterocycle can be pyridine, pyrazole, imidazole, oxazole, isoxazole, thiazole, isothiazole, isoquinoline, pyrrole, pyrazine, pyridazine, pyrimidine, benzimidazole, benzothiazole, indole, triazole, tetrazole, pyran, oxadiazole, triazine, tetrazine, and derivatives thereof.

Rings A and B can be unsubstituted or substituted with one or more alkyl, alkenyl, alkynyl, aryl, cycloalkyl, OR, NR₂, SR, C(O)R, C(O)OR, C(O)NR₂, CN, CF₃, NO₂, SO₂, SOR, SO₃R, halo, aryl, substituted aryl, heteroaryl, substituted heteroaryl or heterocyclic group, where R is independently alkyl, alkenyl, alkynyl, alkylaryl, aryl or cycloalkyl, and additionally, or alternatively, any two adjacent substituted positions of rings A and B together form, independently, a fused 5- or 6-membered cyclic group, wherein the said cyclic group is cycloalkyl, cycloheteroalkyl, aryl, or heteroaryl, and wherein the fused 5- to 6-membered cyclic group may be substituted with one or more of alkyl, alkenyl, alkynyl, alkylaryl, cycloalkyl, haloformyl, hydroxyl, aldehyde, carboxamide, amine, amino, alkoxy, azo, benzyl, carbonate ester, carboxylate, carboxyl, ketamine, isocyanate, isocyanide, isothiocyanate, nitrile, nitro, nitroso, phosphine, phosphate, phosphono, pyridyl, sulfonyl, sulfo, sulfinyl, sulfhydryl, halo, aryl, substituted aryl, heteroaryl, substituted heteroaryl or a heterocyclic group.

C represents the photo-responsive unit. A non-limiting list of examples includes a diarylethene, a spiropyran, a spirooxazine or a rhodamine and the like. Examples of diarylethenes include dithienylethenes and stilbenes. Examples of rhodamine include rhodamine 6G, rhodamine B, rhodamine 123, carboxytetramethylrhodamine (TAMRA), tetramethylrhodamine (TMR), isothiocyanate derivative of tetramethylrhodamine (TRITC), sulforhodamine 101, and rhodamine red. Examples of spiropyrans include those having a structure:

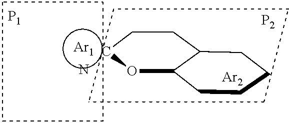

wherein Ar₁ and Ar₂ can represent benzene, naphthalene, anthracene, indolinol, thiophenol rings, or other aromatic rings (including heterocyclic rings) including indolinospiropyran, in which Ar₁ represents indolinol rings. Examples of spirooxazines include the following structures 1-13:

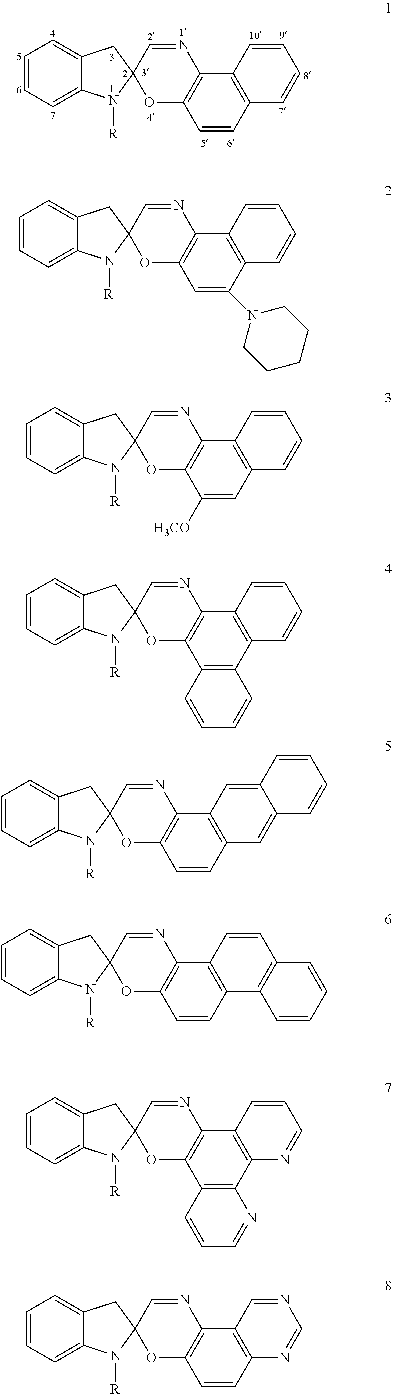

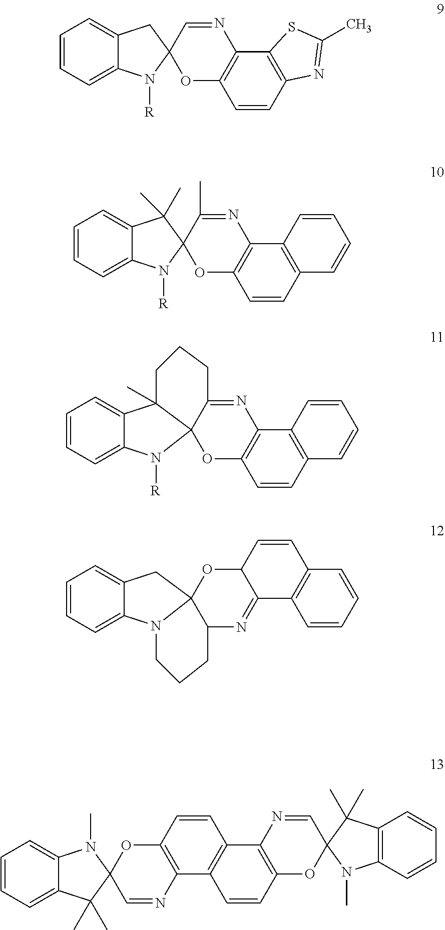

M represents a non-limiting list of metal centers and main group elements including, but not limited to, aluminum, zinc, gallium, indium, rhodium, manganese, nickel, iron, cobalt, copper, ruthenium, platinum, palladium, tin, vanadium, chromium, iridium, gadolinium, boron, beryllium, lanthanum and the like.

L can be independently, but not limited to, alkyl, alkenyl, alkynyl, alkylaryl, cycloalkyl, haloformyl, hydroxyl, aldehyde, carboxamide, amine, amino, alkoxy, benzyl, carbonate ester, carboxylate, carboxyl, ketamine, isocyanate, isocyanide, isothiocyanate, nitrile, nitro, nitroso, phosphine, phosphate, phosphono, pyridyl, sulfonyl, sulfo, sulfinyl, sulfhydryl, halo, aryl, substituted aryl, heteroaryl, substituted heteroaryl or a heterocyclic group, and cyclometalating bidentate ligands which can be, but not limited to, 2-phenylpyridines, phenylisoquinolines, phenylpyrazoles, 7,8-benzoquinolines and derivatives thereof, and non-cyclometalating bidentate ligands which can be, but not limited to, diimine, diamine, diphosphine, dicarboxylate, diketonate, ketoiminate ligands and derivatives thereof, and quinolinato ligands and derivatives thereof. The cyclometalating, non-cyclometalating and quinolinato ligands can be unsubstituted or can be substituted with one or more alkyl, alkenyl, alkynyl, alkylaryl, cycloalkyl, alkoxy, carboxylate, carboxyl, nitro, sulfonyl, SOR, SO₃R, NR₂, SR, CN, CF₃, halo, aryl, substituted aryl, heteroaryl, substituted heteroaryl or a heterocyclic group, where R is independently alkyl, alkynyl, alkynaryl, aryl or cycloalkyl. The cyclometalating and non-cyclometalating ligands can also be extended to tridentate and tetradentate derivatives.

In the present disclosure the following terms are used.

The term “halo” or “halogen” includes a fluorine, chlorine, bromine and iodine. The term “alkyl” as used herein includes either a straight or branched chain alkyl groups. The alkyl groups contain at least one to eighteen or more carbon atoms, including, but not limited to, methyl, ethyl, propyl, isopropyl, butyl, isobutyl, tert-butyl, 3-ethylhexyl and the like. In addition, the alkyl group may be unsubstituted or substituted with one or more substituents including alkenyl, alkynyl, alkylaryl, cycloalkyl, haloformyl, hydroxyl, aldehyde, carboxamide, amine, amino, alkoxy, azo, benzyl, carbonate ester, carboxylate, carboxyl, ketamine, isocyanate, isocyanide, isothiocyanate, nitrile, nitro, nitroso, phosphine, phosphate, phosphono, pyridyl, sulfonyl, sulfo, sulfinyl, sulfhydryl, halo, aryl, substituted aryl, heteroaryl, substituted heteroaryl or a heterocyclic group.

The term “alkenyl” as used herein includes both straight and branched chain alkene radicals of two to eighteen or more carbon atoms. The alkenyl group can be unsubstituted or substituted with one or more substituents including, but not limited to, alkynyl, alkylaryl, cycloalkyl, haloformyl, hydroxyl, aldehyde, carboxamide, amine, amino, alkoxy, azo, benzyl, carbonate ester, carboxylate, carboxyl, ketamine, isocyanate, isocyanide, isothiocyanate, nitrile, nitro, nitroso, phosphine, phosphate, phosphono, pyridyl, sulfonyl, sulfo, sulfinyl, sulfhydryl, halo, aryl, substituted aryl, heteroaryl, substituted heteroaryl or a heterocyclic group.

The term “alkynyl” as used herein includes both straight and branched chain alkyne radicals of two to eighteen or more carbon atoms. The alkynyl group can be unsubstituted or substituted with one or more substituents including, but not limited to, alkyl, alkenyl, alkylaryl, cycloalkyl, haloformyl, hydroxyl, aldehyde, carboxamide, amine, amino, alkoxy, azo, benzyl, carbonate ester, carboxylate, carboxyl, ketamine, isocyanate, isocyanide, isothiocyanate, nitrile, nitro, nitroso, phosphine, phosphate, phosphono, pyridyl, sulfonyl, sulfo, sulfinyl, sulfhydryl, halo, aryl, substituted aryl, heteroaryl, substituted heteroaryl or a heterocyclic group.

The term “alkylaryl” as used herein includes an alkyl group which has an aromatic group as a substituent. The alkynyl group may be unsubstituted or substituted with one or more substituents including, but not limited to, alkyl, alkenyl, cycloalkyl, haloformyl, hydroxyl, aldehyde, carboxamide, amine, amino, alkoxy, azo, benzyl, carbonate ester, carboxylate, carboxyl, ketamine, isocyanate, isocyanide, isothiocyanate, nitrile, nitro, nitroso, phosphine, phosphate, phosphono, pyridyl, sulfonyl, sulfo, sulfinyl, sulfhydryl, halo, aryl, substituted aryl, heteroaryl, substituted heteroaryl or a heterocyclic group.

The term “cycloalkyl” as used herein includes cyclic alkyl groups. Cycloalkyl groups can contain 3 to 7 or more carbon atoms and include cyclopropyl, cyclopentyl, cyclohexyl, and the like. Cycloalkyl groups may be unsubstituted or substituted with one or more substituents including, but not limited to, alkyl, alkenyl, alkylaryl, cycloalkyl, haloformyl, hydroxyl, aldehyde, carboxamide, amine, amino, alkoxy, azo, benzyl, carbonate ester, carboxylate, carboxyl, ketamine, isocyanate, isocyanide, isothiocyanate, nitrile, nitro, nitroso, phosphine, phosphate, phosphono, pyridyl, sulfonyl, sulfo, sulfinyl, sulfhydryl, halo, aryl, substituted aryl, heteroaryl, substituted heteroaryl or a heterocyclic group.

The term “alkoxy” as used herein includes linear or branched alkoxy groups of one to eighteen or more carbon atoms, and can be unsubstituted or substituted with one or more substituents including, but not limited to, alkyl, alkenyl, alkylaryl, cycloalkyl, haloformyl, hydroxyl, aldehyde, carboxamide, amine, amino, alkoxy, azo, benzyl, carbonate ester, carboxylate, carboxyl, ketamine, isocyanate, isocyanide, isothiocyanate, nitrile, nitro, nitroso, phosphine, phosphate, phosphono, pyridyl, sulfonyl, sulfo, sulfinyl, sulfhydryl, halo, aryl, substituted aryl, heteroaryl, substituted heteroaryl or a heterocyclic group.

Aryl alone or in combination includes carbocyclic aromatic systems containing one, two or three rings, wherein each ring may be attached together in a pendant manner or may be fused and can be 5- or 6-membered rings. The aryl rings can be unsubstituted or substituted with one or more substituents including, but not limited to, alkyl, alkenyl, alkylaryl, cycloalkyl, haloformyl, hydroxyl, aldehyde, carboxamide, amine, amino, alkoxy, azo, benzyl, carbonate ester, carboxylate, carboxyl, ketamine, isocyanate, isocyanide, isothiocyanate, nitrile, nitro, nitroso, phosphine, phosphate, phosphono, pyridyl, sulfonyl, sulfo, sulfinyl, sulfhydryl, halo, aryl, substituted aryl, heteroaryl, substituted heteroaryl or a heterocyclic group.

Heteroaryl alone or in combination includes heterocyclic aromatic systems which contain one, two, three or more rings, wherein each ring may be combined in a pendant or fused manner, wherein each ring of the system is a 5- or 6-membered rings.

Heterocyclic and heterocycles refer to a 3- to 7-membered ring containing at least one heteroatom. The heterocyclic rings can be aromatic, including, but not limited to, pyridine, thiophene, furan, pyrazole, imidazole, oxazole, isoxazole, thiazole, isothiazole, isoquinoline, pyrrole, pyrazine, pyridazine, pyrimidine, benzimidazole, benzofuran, benzothiazole, indole, naphthalene, triazole, tetrazole, pyran, thiapyran, oxadiazole, triazine, carbazole, dibenzothiophene, dibenzofuran, indole, and fluorene. The heterocyclic rings can be non-aromatic, including, but not limited to, aziridine, oxirane, thiirane, oxaziridine, dioxirane, azetidine, oxetane, thietane, diazetidine, dioxetane, dithietane, tetrahydrofurane, thiolane, borolane, phospholane, arsolane, stibolane, bismolane, silane, stannolane, piperazine, piperidine, and pyrrolidine. Heterocyclic rings can be unsubstituted or substituted, which can include, but not limited to, alkyl, alkoxy, aryl.

Cyclometalating bidentate ligand is a term well known in the art and includes, but not limited to, 2-phenylpyridine (ppy), 2-(p-tolyl)pyridine (ptpy), 4-(2-pyridyl)benzaldehyde (pba), 2-(2,4-difluorophenyl)pyridine (fppy), 4-pyridin-2-ylbenzoic acid, 3-pyridin-2-ylbenzoic acid, 2-methyl-6-phenylpyridine, 3-methyl-2-phenylpyridine, 4-methyl-2-phenylpyridine, 5-methyl-2-phenylpyridine, 2-phenylpyridine-3-carboxylic acid, 2-phenylpyridine-4-carboxylic acid, 6-phenylpyridine-3-carboxylic acid, 2,3-diphenylpyridine, 2,4-diphenylpyridine, 2,5-diphenylpyridine, phenylpyrazole (ppz), 3-methyl-1-phenyl-1H-pyrazole (mppz), 7,8-benzoquinoline (bzq), 2-phenylquinoline (pq), 1-phenylisoquinoline (piq), 2-phenylquinoline, 3-phenylisoquinoline, 3,4-diphenylisoquinoline and 2-(benzimidazol-2-yl)quinolone (biq).

Non-cyclometalating bidentate ligand is a term well known in the art and includes, but not limited to, 2,2′-bipyridine (bpy), 4-chloro-2,2′-bipyridine (4-Cl-bpy), 4-carboxy-2,2′-bipyridine, 4,4′-dimethyl-2,2′-bipyridine (4,4′-Me₂-bpy), 4,4′-diphenyl-2,2′-bipyridine (4,4′-Ph₂-bpy), 4,4′-dicarboxy-2,2′-bipyridine, 5,5′-bis(ethoxycarbonyl)-2,2′-bipyridine, 5-chloro-2,2′-bipyridine, 6-bromo-2,2′-bipyridine, 1,10-phenanthroline (phen), 4-chloro-1,10-phenanthroline (4-CI-phen), 4-methyl-1,10-phenanthroline (4-Me-phen), 5-bromo-1,10-phenanthroline (5-Br-phen), 5-phenyl-1,10-phenanthroline (5-Ph-phen), 5-nitro-1,10-phenanthroline (5-NO₂-phen), 4,7-dimethyl-1,10-phenanthroline (4,7-Me₂-phen), 4,7-diphenyl-1,10-phenanthroline (4,7-Ph₂-phen), 5,6-dimethyl-1,10-phenanthroline (5,6-Me₂-phen), 3,4,7,8-tetramethyl-1,10-phenanthroline (3,4,7,8-Me₄-phen), 3,8-dibromo-1,10-phenanthroline (3,8-Br₂-phen), 5,6-dibromo-1,10-phenanthroline (5,6-Br₂-phen), 2,9-dichloro-1,10-phenanthroline (2,9-Cl₂-phen), 2,9-dimethyl-4,7-diphenyl-1,10-phenanthroline (2,9-Me₂-4,7-Ph₂-phen), dipyrido[3,2-a:2′,3′-c]phenazine (dppz), benzo[i]dipyrido[3,2-a:2′,3′-c]phenazine (dppn), 2,2′-bis(diphenylphosphino)-1,1′-binaphthyl (BINAP), 2,2′-bis(di-p-tolylphosphino)-1,1′-binaphthyl, 2,2′-bis[di(3,5-xylyl)phosphino]-1,1′-binaphthyl, 2,2′-bis(diphenylphospino)-5,5′,6,6′,7,7′,8,8′-octahydro-1,1′-binaphthyl, 1,2-bis(diphenylphosphino)ethane, 1,3-bis(diphenylphosphino)propane, bis[(2-diphenylphosphino)phenyl]ether, 4,5-bis(diphenylphosphino)-9,9-dimethyl-xanthene, ethylenediamine, oxalate, acetylacetonate, hexafluoroacetylacetonate, 1,3-diphenyl-1,3-propanedionate.

Quinolinato ligand is a term well known in the art and includes, but not limited to, 8-hydroxyquinoline, 5-chloro-8-hydroxyquinoline, 7-bromo-8-hydroxyquinoline, 2-amino-8-quinolinol, 2-methyl-8-quinolinol, 5,7-dimethyl-8-quinolinol, 8-hydroxyquinoline-7-carbaldehyde, 8-hydroxy-2-quinolinecarboxylic acid 8-hydroxyquinoline-5-sulfonic acid monohydrate, 2-benzyl-8-hydroxyquinoline and 8-mercaptoquinoline.

Benzene includes substituted or unsubstituted benzene.

Pyridine includes substituted or unsubstituted pyridine.

Thiophene includes substituted or unsubstituted thiophene.

Furan includes substituted or unsubstituted furan.

Fused-thiophene includes substituted or unsubstituted fused-thiophene.

Pyrazole includes substituted or unsubstituted pyrazole.

Pyrimidine includes substituted or unsubstituted pyrimidine.

Pyrrole includes substituted or unsubstituted pyrrole.

Benzimidazole includes substituted or unsubstituted benzimidazole.

Benzofuran includes substituted or unsubstituted benzofuran.

Benzothiazole includes substituted or unsubstituted benzothiazole.

Indole includes substituted or unsubstituted indole.

Naphthalene includes substituted or unsubstituted naphthalene.

Anthracene includes substituted or unsubstituted anthracene.

Pyrene includes substituted or unsubstituted pyrene.

Thiazole includes substituted or unsubstituted thiazole.

Pyran includes substituted or unsubstituted pyran.

Thiapyran includes substituted or unsubstituted thiapyran.

Carbazole includes substituted or unsubstituted carbazole.

Dibenzothiophene includes substituted or unsubstituted dibenzothiophene.

Dibenzofuran includes substituted or unsubstituted dibenzofuran.

Fluorene includes substituted or unsubstituted fluorene.

The invention is illustrated by the following non-limiting examples. It is to be understood that changes and variations can be made therein without deviating from the scope and the spirit of the invention as hereinafter claimed. It is also understood that various theories as to why the invention works are not intended to be limiting. The compounds described herein are represented throughout by their monomeric structure. As is well known to those in the art, the compounds may also be present as dimers, trimers, larger oligomers, dendrimers, or polymers.

In some embodiments, the photo-responsive coordination compounds of formula (I) are prepared in high purity. High purity means one of at least 90% by weight pure, at least 95% by weight pure, at least 99% by weight pure, or at least 99.9% by weight pure.

The photo-responsive coordination compounds can be used to form thin films by spin-coating, spray-coating, dip-coating, layer-by-layer deposition, ink-jet printing, 3D printing, or other known suitable fabrication methods and be subjected to achieve photo-responsive electron-transporting functions and applications in organic resistive memory devices. Referring to FIG. 1, one example of a structure of a device for measuring electrical conductivity of the photo-responsive coordination compounds is shown in order: aluminum 10/LiF 11/active layer 12/LiF 13/compound 14/ITO coated glass 15, in which the active layer is formed by mixing the photo-responsive coordination compound as a dopant into a host complex. Suitable host materials should be selected to ensure an efficient energy transfer between the host material and the dopant material. Examples of desirable hosts are m-(N,N′-dicarbazole)benzene (mCP), 4,4′-bis(carbazol-9-yl)-biphenyl (CBP), 4,4′,4″-tris(carbazol-9-yl)triphenylamine (TCTA), 3-(4-biphenylyl)-4-phenyl-5-tert-butylphenyl-1,2,butylphenyl-1,2,4-triazole (TAZ), p-bis(triphenylsilyl)benzene (UGH2), and PVK. LiF is deposited on the ITO coated glass or on the active layer by thermal deposition. A layer of aluminum is then thermally deposited with shadow mask on the LiF layer. In some embodiments, the electrical conductivity of the photo-responsive coordination compound dramatically increases by two-fold after light-irradiation. Unlike the conventional method by modulating electrical conductivity through the reconstruction of molecular structure of the target compound, an integration of photochromic unit into the coordination compound in the invention provides a direct and simple way to enhance the versatility of the corresponding coordination compounds for the application in organic electronics.

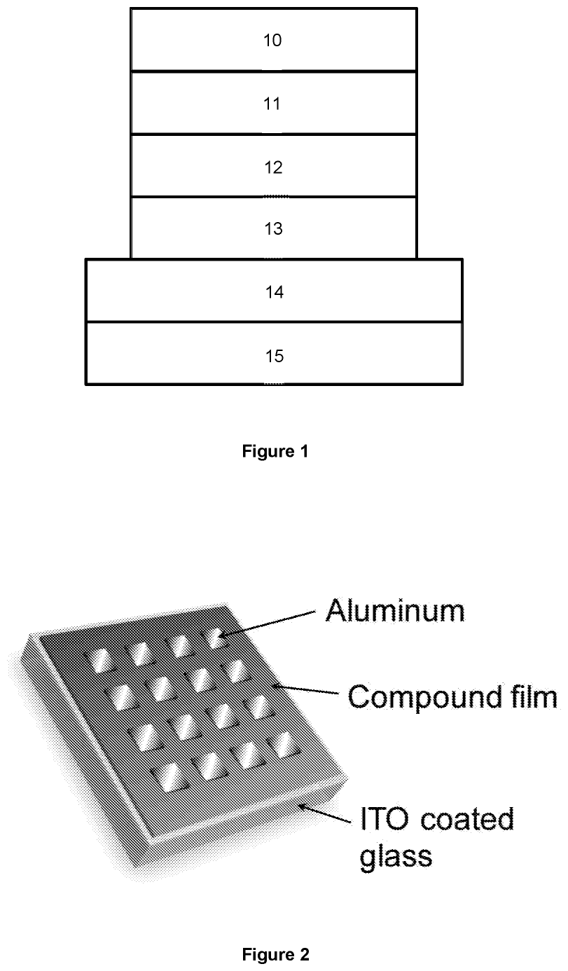

Meanwhile, the photo-responsive coordination compounds have been shown to serve as electroactive components for the applications in organic resistive memory devices. The typical structure of an organic resistive memory device is in the order shown in FIG. 2: aluminum 20/active layer 21/ITO coated glass 22, in which the photo-responsive compound serves as active layer material and is formed by spin-coating onto the ITO coated glass and covered by a layer of aluminum cathode prepared by thermal deposition with shadow mask on the former. The size of the ITO coated glass is 2 cm×2 cm, in which 400 individual devices have been prepared simultaneously. Particularly, memory performance has been obtained after photo-irradiation onto the active layer. A high ON/OFF ratio of up to 10⁴, a threshold voltage of about 3.5 V and a long retention time over 10⁴ s have been achieved. Notably, the present invention represents the first example of resistive memory device based on coordination compound with photochromic motif, exhibiting unique photo-responsive memory performance reported so far.

The following examples illustrate the subject invention. Unless otherwise indicated in the following examples and elsewhere in the specification and claims, all parts and percentages are by weight, all temperatures are in degrees Centigrade, and pressure is at or near atmospheric pressure.

### Example 1

### Synthesis and Characterization of Compounds 1-4

Compounds 1-4 were synthesized according to the following methodology. The desired compounds were synthesized by the coordination of the diarylethene-containing 8-hydroxyquinolines to the Al(III) center. For example, triethylaluminum in heptane was added to a solution of 5-(4-(2-(2,5-dimethylthiophen-3-yl)benzo[b]thiophen-3-yl)-5-methylthiophen-2-yl)quinolin-8-ol in toluene, (1 M) (0.50 mmol). The mixture was stirred overnight and the solvent was removed under vacuum afterwards. The aluminum compound was purified by recrystallization with dichloromethane-ether to afford the Al(III) compounds.

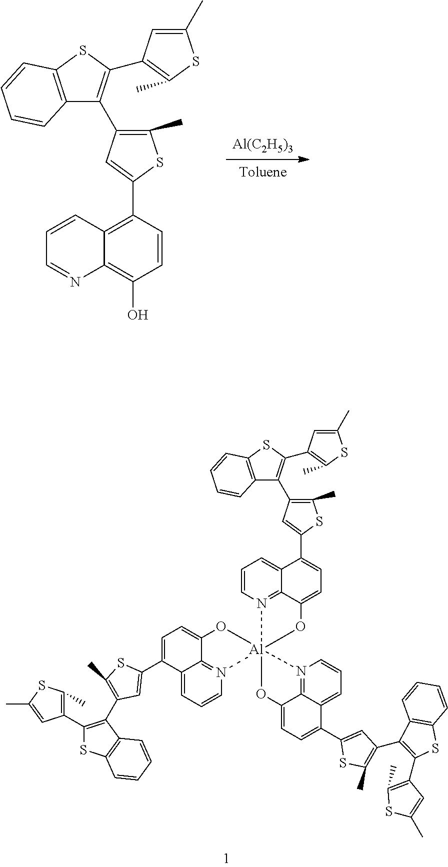

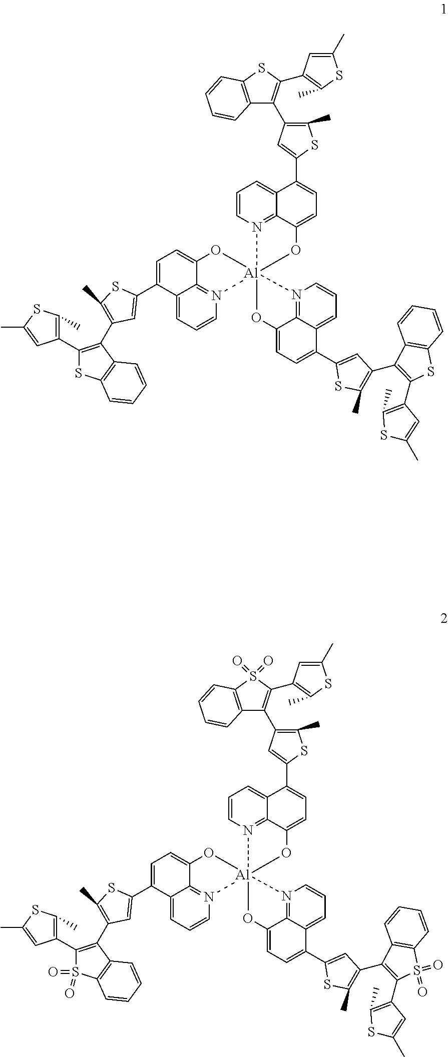

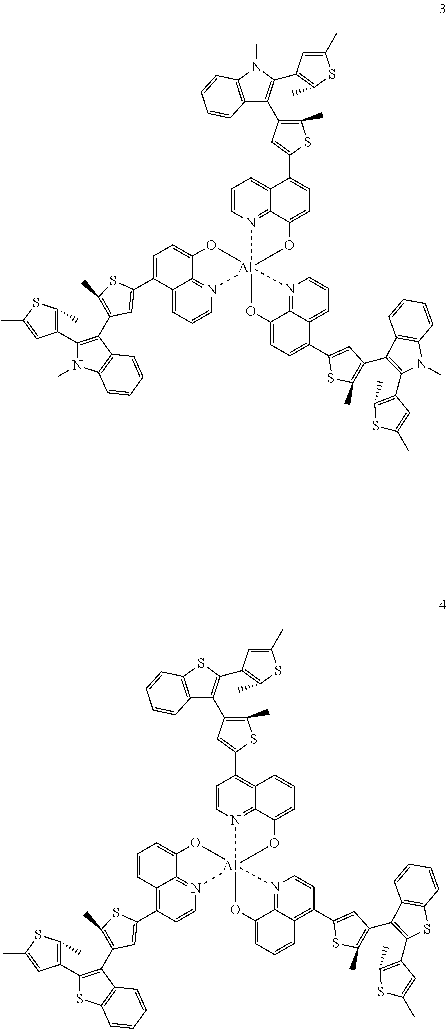

The characteristic spectroscopic properties of compounds 1-4 are as follows:

Compound 1

Yield: 0.60 g, 0.40 mmol, 81%. ¹H NMR (400 MHz, [D₆]DMSO, 298 K, δ/ppm): δ 2.04-2.08 (m, 9H, —CH₃), 2.14-2.16 (m, 9H, —CH₃), 2.35-2.37 (s, 9H, —CH₃), 6.68-6.64 (m, 3H, thienyl), 6.82-6.86 (m, 3H, quinolinyl), 6.98-7.00 (m, 3H, thienyl), 7.42-7.46 (m, 8H, phenyl and quinolinyl), 7.61-7.68 (m, 8H, phenyl and quinolinyl), 8.01 (m, 3H, phenyl), 8.35-8.43 (m, 3H, quinolinyl), 8.83 (s, 1H, quinolinyl), 8.90 (s, 1H, quinolinyl). HRMS (positive ESI) calcd. for C₈₄H₆₀AlN₃O₃S₉: m/z 1473.1936; found: 1473.1919 [M]⁺. Elemental analyses, found (%): C, 65.42, H, 4.13, N, 2.70; calcd (%) for C₈₄H₆₀AlN₃O₃S₉CH₂Cl₂: C, 65.45, H, 4.01, N, 2.69.

Compound 2

Yield: 0.64 g, 0.41 mmol, 82%. ¹H NMR (400 MHz, [D₆]DMSO, 298 K, δ/ppm): δ 2.01-2.08 (m, 9H, —CH₃), 2.11-2.16 (m, 9H, —CH₃), 2.41-2.44 (s, 9H, —CH₃), 6.85-6.89 (m, 3H, thienyl), 7.02 (d, J=8.0 Hz, 3H, quinolinyl), 7.11-7.17 (m, 3H, thienyl), 7.40-7.42 (m, 3H, phenyl), 7.56-7.74 (m, 12H, phenyl and quinolinyl), 8.01 (m, 4H, phenyl and quinolinyl), 8.44-8.57 (m, 3H, quinolinyl), 8.73 (d, J=4.2 Hz, 1H, quinolinyl), 8.87 (d, J=4.2 Hz, 1H, quinolinyl). HRMS (positive ESI) calcd. for C₈₄H₆₀AlN₃O₉S₉: m/z 1569.1631; found: 1569.1610 [M]*. Elemental analyses, found (%): C, 58.43, H, 3.94, N, 2.63; calcd (%) for C₈₄H₆₀AlN₃O₉S₉ 2.5CH₂Cl₂: C, 58.26, H, 3.67, N, 2.36.

Compound 3

Yield: 0.59 g, 0.41 mmol, 80%. ¹H NMR (400 MHz, [D₆]DMSO, 298 K, δ/ppm): δ 1.93-1.95 (m, 9H, —CH₃), 2.21-2.23 (m, 9H, —CH₃), 2.41-2.44 (s, 9H, —CH₃), 3.64 (s, 9H, —CH₃), 6.77-6.81 (m, 6H, phenyl and thienyl), 6.96-6.98 (m, 3H, thienyl), 7.12 (t, J=6.8 Hz, 3H, phenyl), 7.23 (t, J=6.8 Hz, 3H, phenyl), 7.41-7.53 (m, 12H, phenyl and quinolinyl), 7.70 (s, 1H, quinolinyl), 8.30 (s, 3H, quinolinyl), 8.69 (s, 1H, quinolinyl), 8.84 (s, 1H, quinolinyl). HRMS (positive ESI) calcd. for C₈₇H₆₉AlN₆O₃S₆: m/z 1464.3571; found: 1464.3437 [M]⁺. Elemental analyses, found (%): C, 69.62, H, 5.11, N, 5.34; calcd (%) for C₈₇H₆₉AlN₆O₃S₆2H₂O: C, 69.57, H, 4.90, N, 5.60.

Compound 4

Yield: 0.60 g, 0.42 mmol, 83%. ¹H NMR (400 MHz, [D₆]DMSO, 298 K, δ/ppm): δ 2.00-2.04 (m, 9H, —CH₃), 2.14-2.19 (m, 9H, —CH₃), 2.34-2.36 (s, 9H, —CH₃), 6.65-6.81 (m, 6H, phenyl and thienyl), 6.94-6.96 (m, 3H, thienyl), 7.16-7.27 (m, 6H, phenyl), 7.31-7.58 (m, 12H, phenyl and quinolinyl), 7.68 (s, 1H, quinolinyl), 7.97-8.01 (m, 1H, quinolinyl), 8.59 (s, 1H, quinolinyl), 8.72 (s, 1H, quinolinyl). HRMS (positive ESI) calcd. for C₈₄H₆₁AlN₃O₃S₉: m/z 1474.2015; found: 1474.2005 [M+H]⁺. Elemental analyses, found (%): C, 66.51, H, 4.40, N, 2.78; calcd (%) for C₈₄H₆₀AlN₃O₃S₉2H₂O: C, 66.77, H, 4.27, N, 2.78.

### Example 2

### Photochromic Properties

A solution sample of the compound was degassed on a high vacuum line in a degassing cell with a 10 cm³ Pyrex round-bottom flask connected by a side-arm to a 1-cm quartz fluorescence cuvette and was sealed from the atmosphere by a Rotaflo HP6/6 quick-release Teflon stopper. The solution sample was rigorously degassed with no fewer than four freeze-pump-thaw cycles prior to the measurements.

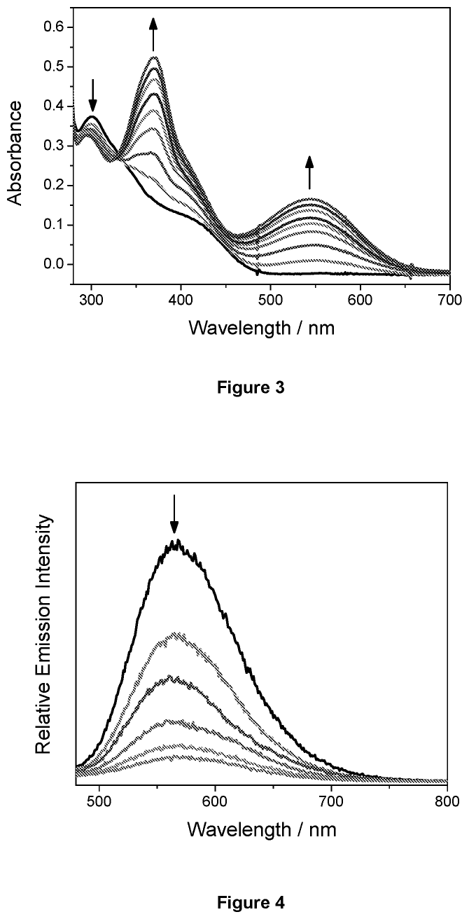

The solution sample was irradiated at the UV absorption band, whereby the initial pale yellow solution turned into various colors. The colored state was thermally stable. Then, it was irradiated with visible light, whereby the solution was back to the original color. The UV-vis absorbance changes of the compounds were capable of undergoing reversible cycles. FIG. 3 shows the UV-vis absorption spectral changes of 2 in degassed benzene upon excitation at 300 nm. The electronic absorption data of the open forms and closed forms are summarized in Table 1. In addition to the UV-vis spectral change, the emission intensity would decrease upon photocyclization of the Alq₃ compounds (FIG. 4), rendering the compounds photo-switchable. The quantum yields for both photocyclization and photocycloreversion of the photochromic compounds are summarized in Table 2. The conversion at photostationary state is also summarized in Table 2.

TABLE 1
Electronic absorption data for compounds 1-4 in benzene solution at 298K

| | | Absorption |
|---|---|---|
| Compound | Configuration | λ_{abs}/nm (ε × 10⁻³/dm³mol⁻¹cm⁻¹) |
| 1 | Open form | 298 (62.8), 421 (10.5) |
| | Closed form | 352 (113.0), 407 (33.5), 593 (57.2) |
| 2 | Open form | 300 (25.2), 413 (7.7) |
| | Closed form | 293 (17.2), 372 (38.4), 412 sh (16.5), |
| | | 543 (14.1) |
| 3 | Open form | 298 (53.1), 424 (7.9) |
| | Closed form | 358 (161.2), 415 sh (34.2), 647 (54.1) |
| 4 | Open form | 307 (55.3), 343 sh (24.9), 428 (12.2) |
| | Closed form | 303 (54.2), 347 sh (27.8), 426 (12.0), |
| | | 626 (4.9) |

TABLE 2
Photochemical quantum yields and conversion at photostationary state
determined in degassed benzene solution at 298K

| | Photochemical Quantum Yield/ϕ^{[a]} | | Conversion at PSS |
|---|---|---|---|
| Compound | Photocyclization^{[b]} | Photocycloreversion^{[c]} | % |
| 1 | 0.012 | 0.005 | 45 |
| 2 | 0.107 | 0.035 | 89 |
| 3 | 0.006 | 0.004 | 36 |
| 4 | 0.009 | 0.005 | 24 |

^{[a]}Data obtained with an uncertainty of ±10%.
^{[b]}Data obtained using 316 nm as the excitation source.
^{[c]}Data obtained using 600 nm as the excitation source.

### Example 3

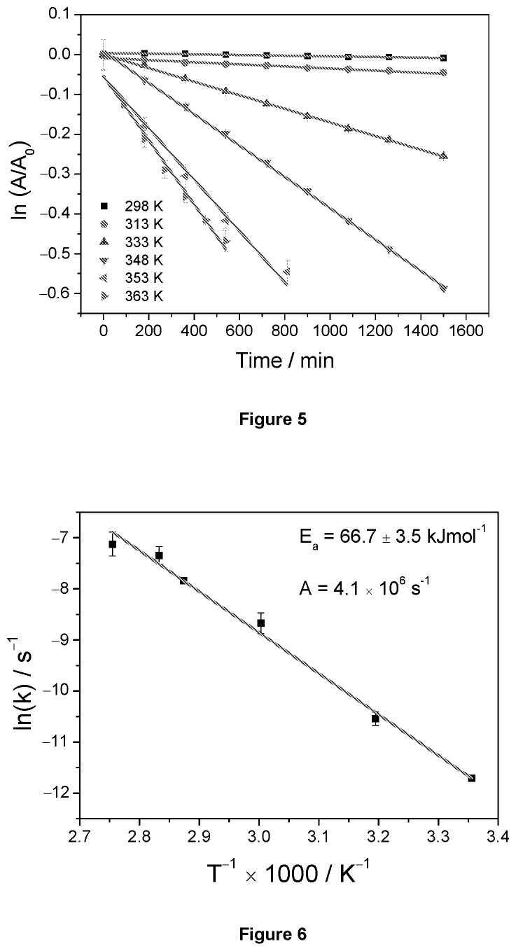

Compound 2 was used to demonstrate the thermal stability of the closed form of the photochromic compounds by measuring the absorbance decay at different temperatures in the dark (FIG. 5). By plotting the rates of thermal backward reaction at different temperature against the temperature, the Arrhenius plot (FIG. 6) could be obtained, which could be used to determine the activation energy (66.7 kJ mol⁻¹) and the pre-exponential factor (4.1×10⁶ s⁻¹) of the thermal cycloreversion of compound 2.

### Example 4

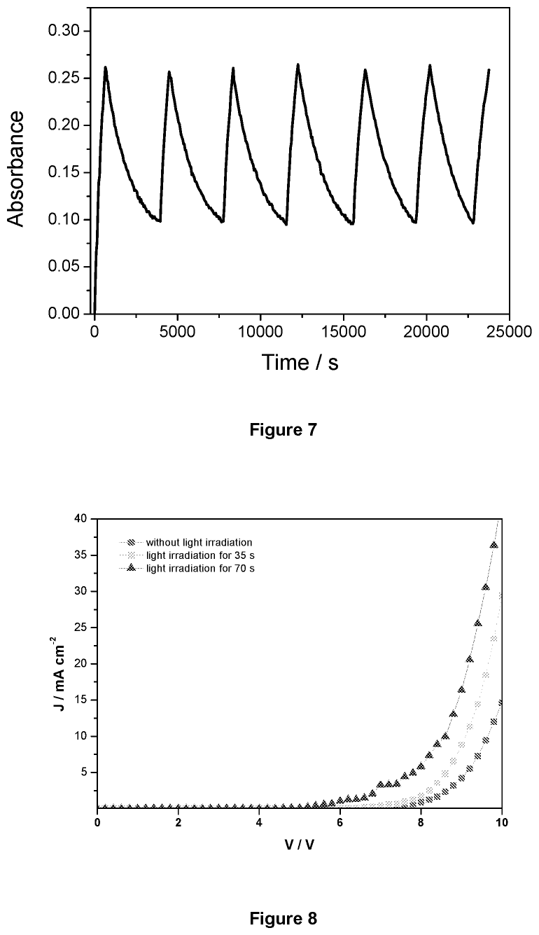

Fatigue resistance represents another important parameter that is commonly used to evaluate the performance of photochromic materials. Photochromic materials could lose their photochromic reactivities through side-reactions of the closed form. The fatigue resistance of the compound could be demonstrated by alternate excitation at the absorption bands of the open form and the closed form of the compounds and monitoring the UV-vis absorption changes at a selected wavelength. Compound 2 was used to demonstrate the fatigue resistance of the photochromic compounds, as depicted in FIG. 7.

### Example 5

### Electrochemical Properties

To probe the electrochemical properties, cyclic voltammetry was carried out in a three-electrode cell with 0.1 M ⁿBu₄NPF₆ as the supporting electrolyte in dichloromethane for compounds 1 to 4. The ferrocenium/ferrocene couple (Fc⁺/Fc) was used as the internal reference.

Compounds 1-4 show several irreversible oxidation waves at around +0.95 to +1.99 V versus standard calomel electrode (SCE). For the reduction process, one or two quasi-reversible reduction couples as well as reduction waves at about −1.57 to −1.82 V versus SCE can be observed. Upon structural modification of the 8-hydroxyquinoline ligands on the peripheral photochromic units, no considerable shifts of the first oxidation wave can be observed. As for compounds 1 and 3, which bear no substituents on the pyridyl side of the ligands, similar reduction potential is observed. Meanwhile, a less negative reduction couple at about −1.57 V is found in compound 2. Compound 4 also exhibits a reduction potential with less negative value, indicating that substitution of the dithienylethene unit on the pyridyl side possesses a significant influence on the electronic properties of the aluminum(III) compound. The highest occupied molecular orbital (HOMO) levels and the lowest unoccupied molecular orbital (LUMO) levels of compounds 1-4 had been determined by using ferrocene as the reference. The LUMO levels of all the compounds are found to be in the range of −2.52 to −2.77 eV, while the HOMO levels of all the compounds are found to range from −5.29 to −5.50 eV. The electrochemical data are summarized in Table 3.

TABLE 3
Electrochemical data in dichloromethane solution at 298K

| | | Reduction^{[a]} | | |
|---|---|---|---|---|
| Compound | | E_{1/2}/V^{[c]} | | |
| Oxidation^{[a]} | | vs. SCE | | |
| Eₚₐ/V | | [E_{pc}/V | HOMO^{[e} | LUMO^{[e]} |
| vs. SCE^{[b]} | | vs. SCE^{[d]} | [eV] | [eV] |
| 1 | +0.97, +1.13, | [−1.81] | −5.31 | −2.53 |
| | +1.71 | | | |
| 2 | +1.04, +1.25, | −1.57 (60), | −5.38 | −2.77 |
| | +1.99 | −1.77 (60) | | |
| 3 | +0.95, +1.39, | [−1.82] | −5.29 | −2.52 |
| | +1.48, +1.77 | | | |
| 4 | +1.16, +1.41, | −1.58 (80), | −5.50 | −2.76 |
| | +1.79 | −1.74 (60) | | |

^{[a]}0.1M ⁿBu₄NPF₆ (TBAH) as supporting electrolyte at room temperature; scan rate 100 mV s⁻¹.
^{[b]}Anodic peak potential for the irreversible oxidation wave.
^{[c]}E_{1/2} = (Eₚₐ + E_{pc})/2; Eₚₐ and E_{pc} are peak anodic and peak cathodic potentials, respectively.
^{[d]}Cathodic peak potential for the irreversible reduction wave.
^{[e]}The energy level is determined with reference to the HOMO level of ferrocene (−4.8 eV vs. vacuum level).

### Example 6

A device for measuring the electrical conductivity according to an embodiment of the invention was constructed in the following manner:
a) A transparent anode ITO coated borosilicate glass substrate (38 mm×38 mm) with sheet resistance of 30Ω per square was ultrasonicated in the commercial detergent Decon 90, rinsed in deionized water having a resistivity of 18.2 mega-ohm for 15 minutes, and then dried in an oven at 120° C. for an hour. The substrate was next subjected to an UV-ozone treatment in a Jelight 42-220 UVO cleaner equipped with a mercury grid lamp for 15 minutes.
b) A 5-nm thick LiF layer was deposited by thermal evaporation on the ITO coated glass substrate of step a.
c) A 60-nm thick active layer was spin-coated by using a Laurell WS-400Ez-6NPP-Lit2 single wafer spin processor at 6000 rpm for 30 seconds onto LiF layer of step b, and baked at 80° C. for 10 minutes in air, in which compound 2 was doped into the host material MCP layer at a concentration of 20 wt %.
d) A 5-nm thick LiF layer and a 100-nm thick Al layer were deposited by thermal evaporation on the active layer of step c to form an electron-injecting cathode.

LiF and Al were prepared by thermal evaporation from tantalum boats by applying current through the tantalum boats. Deposition rates were monitored with a quartz oscillation crystal together with a Sigma SQM-242 quartz crystal card and controlled at 0.1-0.2 nm s⁻¹ for both organic and metal layers. J-V characteristics of the devices was measured with a programmable Keithley model 2420 source meter under ambient air conditions.

### Example 7

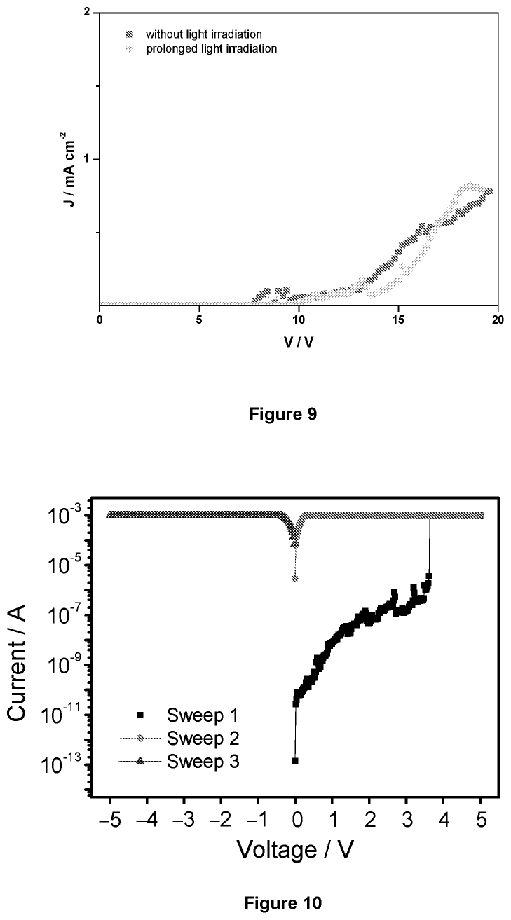

FIG. 8 shows the J-V curves of the devices doped with compound 2. Upon light-irradiation, a significant lowering of the driving voltage can be observed as compared to the device without any exposure to UV light. The current density at particular voltage was also found to be higher for the devices after photo-irradiation upon comparing to those without any light exposure. Notably, the extent of the lowering of the driving voltage becomes more significant upon lengthening the time of light exposure. In addition, it is found that the electrical conductivity of compound 2 has been improved to 7.8×10⁻⁶ mS cm⁻¹ after photo-irradiation, which is a recognizable improvement from the non-irradiated conditions (i.e. 3.7×10⁻⁶ mS cm⁻¹). However, as shown in FIG. 9, there was no significant difference between the J-V curves obtained from the devices containing the original Alq₃ compound with or without exposure to UV light. Such two-fold increment in electrical conductivity in the diarylethene-containing Alq₃ compounds demonstrates that the coordination compounds are promising candidates for photo-responsive electron-transporting materials.

### Example 8

A memory device according to an embodiment of the invention was constructed in the following manner:
(a) A transparent anode ITO coated borosilicate glass substrate (2 cm×2 cm) was ultrasonicated successively with deionized water having a resistivity of 18.2 mega-ohm, analytical grade acetone, analytical grade isopropanol, absolute ethanol for 15 minutes and then dried in an oven at 120° C. for an hour.
(b) A 300 μL toluene solution of compound 2 was spin-coated onto the ITO substrate of step a in a 2-step spinning mode, 500 rpm for 9 seconds followed by 2000 rpm for 30 seconds;
(c) The substrate was placed in oven at 75° C. for 30 min to remove the solvent residue;
(d) The substrate was put into a vacuum chamber, and the chamber was pumped down from 1 bar to 5×10⁻⁶ mbar;
(e) An aluminum layer was deposited by thermal evaporation onto the compound 2 of step b to form a cathode. J-V characteristics of the memory devices were measured with a programmable Keithley model 4200 power source in a four-probe station. 400 devices were fabricated on each ITO glass substrate and the active area of each cell was 0.25 mm².

### Example 9

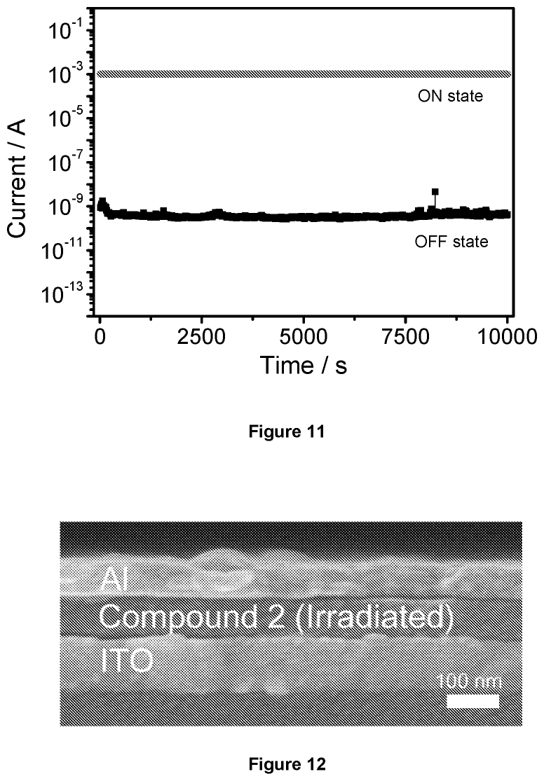

The J-V characteristics of compound 2 of the as-fabricated memory device without and with UV exposure were investigated. For the device without any exposure to light-irradiation, there was no increase in current and a high-resistance state (OFF state) was retained under a voltage ranging from 0 to +5 V. Interestingly, after photo-irradiation, an abrupt increase in the current at the switching threshold voltage at about 3.5 V was observed as shown in FIG. 10. This process demonstrates the transition from a low-conductivity state to a high-conductivity state (ON state) with a high ON/OFF ratio of over 10⁴ from 10⁻⁷ to 10⁻³ A. The device remained in the ON state when the voltage sweep was repeated (sweep 2), indicating that the ON state is retained and memory effect is obtained. The memory device could not be returned to the OFF state by the application of a reverse bias of the same magnitude that it had been switched ON (sweep 3). The stability of the device under a constant stress of 1 V is shown in FIG. 11. No significant degradation was observed in current for ON and OFF states for at least 10000 seconds during the measurement, suggesting that the device exhibits a precise control of the ON and OFF state with low misreading rate. The film thicknesses of compound 2 and aluminum were found to be about 77 nm and 78 nm, respectively, by using SEM to observe a cross section of the device, as shown in FIG. 12. These findings indicate the uniqueness of the diarylethene-containing Alq₃ compounds for the fabrication of organic memory devices, of which the performance is photo-controllable.

These examples should not be construed as limiting the scope of the invention, but as providing illustrations of some of the embodiments of the invention. It is being understood that changes and variations can be made therein without deviating from the scope and the spirit of the invention as hereinafter claimed.

With respect to any figure or numerical range for a given characteristic, a figure or a parameter from one range may be combined with another figure or a parameter from a different range for the same characteristic to generate a numerical range.

Other than in the operating examples, or where otherwise indicated, all numbers, values and/or expressions referring to quantities of ingredients, reaction conditions, etc., used in the specification and claims are to be understood as modified in all instances by the term “about”.

While the invention is explained in relation to certain embodiments, it is to be understood that various modifications thereof will become apparent to those skilled in the art upon reading the specification. Therefore, it is to be understood that the invention disclosed herein is intended to cover such modifications as fall within the scope of the appended claims.

1. A photo-responsive coordination compound comprising a chemical structure represented by formula (I): 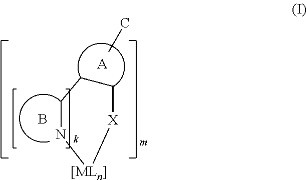
wherein
a) X is oxygen, sulphur, selenium, NR or PR where R is an alkyl, alkylaryl, cycloalkyl, alkoxy, benzyl, aryl, substituted aryl, heteroaryl, substituted heteroaryl or a heterocyclic group;
b) A is cyclic structure derivative of substituted or unsubstituted arene or heteroarene;
c) B, which is optionally fused with or singly-bonded to A, is cyclic structure derivative of substituted or unsubstituted heterocyclic group containing one or more nitrogen atoms;
d) C is a photochromic unit;
e) [MLₙ] represents the coordination unit containing a metal or main group element M and L is a ligand;
f) k is a number of rings in the cyclic structure derivatives and k is an integer from 0 to 2;
g) n is a number of ligands and n is an integer from 0 to 4; and
h) m is a number of the photochromic ligand and m is an integer from 1 to 4.

2. The photo-responsive compound according to claim 1, wherein ring A is a cyclic structure derivative where the cyclic structure is independently selected from a 5- or 6-membered arene or heteroarene, wherein the arene is one or more of benzene, naphthalene, anthracene, pyrene, fluorene and derivatives thereof, and the heteroarene can be pyridine, pyrazole, imidazole, oxazole, isoxazole, thiazole, isothiazole, isoquinoline, pyrrole, pyrazine, pyridazine, pyrimidine, benzimidazole, benzothiazole, indole, triazole, tetrazole, pyran, oxadiazole, triazine, tetrazine, and derivatives thereof.

3. The photo-responsive compound according to claim 1, wherein ring B is a cyclic structure derivative where the cyclic structure is independently selected from a 5- or 6-membered nitrogen-containing heteroarene or heterocycle, wherein the heteroarene or heterocycle is one or more of pyridine, pyrazole, imidazole, oxazole, isoxazole, thiazole, isothiazole, isoquioline, pyrrole, pyrazine, pyridazine, pyrimidine, benzimidazole, benzothiazole, indole, triazole, tetrazole, pyran, oxadiazole, triazine, tetrazine, and derivatives thereof.

4. The photo-responsive compound according to claim 1, wherein rings A and B are unsubstituted or substituted with one or more alkyl, alkenyl, alkynyl, aryl, cycloalkyl, OR, NR₂, SR, C(O)R, C(O)OR, C(O)NR₂, CN, CF₃, NO₂, SO₂, SOR, SO₃R, halo, aryl, substituted aryl, heteroaryl, substituted heteroaryl or heterocyclic group, where R is independently alkyl, alkenyl, alkynyl, alkylaryl, aryl or cycloalkyl, and additionally, or alternatively, any two adjacent substituted positions of rings A and B together form, independently, a fused 5- or 6-membered cyclic group, wherein the said cyclic group is cycloalkyl, cycloheteroalkyl, aryl, or heteroaryl, and wherein the fused 5- to 6-membered cyclic group may be substituted with one or more of alkyl, alkenyl, alkynyl, alkylaryl, cycloalkyl, haloformyl, hydroxyl, aldehyde, carboxamide, amine, amino, alkoxy, azo, benzyl, carbonate ester, carboxylate, carboxyl, ketamine, isocyanate, isocyanide, isothiocyanate, nitrile, nitro, nitroso, phosphine, phosphate, phosphono, pyridyl, sulfonyl, sulfo, sulfonyl, sulfhydryl, halo, aryl, substituted aryl, heteroaryl, substituted heteroaryl or a heterocyclic group.

5. The photo-responsive compound according to claim 1, wherein M is a metal center selected from one or more of aluminum, zinc, gallium, indium, rhodium, manganese, nickel, iron, cobalt, copper, ruthenium, platinum, palladium, tin, vanadium, chromium, iridium, gadolinium, boron, beryllium, and lanthanum.

6. The photo-responsive compound according to claim 1, wherein L is independently alkyl, alkenyl, alkynyl, alkylaryl, cycloalkyl, haloformyl, hydroxyl, aldehyde, carboxamide, amine, amino, alkoxy, benzyl, carbonate ester, carboxylate, carboxyl, ketamine, isocyanate, isocyanide, isothiocyanate, nitrile, nitro, nitroso, phosphine, phosphate, phosphono, pyridyl, sulfonyl, sulfo, sulfinyl, sulfhydryl, halo, aryl, substituted aryl, heteroaryl, substituted heteroaryl or a heterocyclic group, and cyclometalating bidentate ligands which are optionally selected from 2-phenylpyridines, phenylisoquinolines, phenylpyrazoles, 7,8-benzoquinolines and derivatives thereof, and non-cyclometalating bidentate ligands which are optionally selected from diimine, diamine, diphosphine, dicarboxylate, diketonate, ketoiminate ligands and derivatives thereof, and quinolinato ligands and derivatives thereof, the cyclometalating, non-cyclometalating and quinolinato ligands are one or more of unsubstituted or substituted with one or more alkyl, alkenyl, alkynyl, alkylaryl, cycloalkyl, alkoxy, carboxylate, carboxyl, nitro, sulfonyl, SOR, SO₃R, NR₂, SR, CN, CF₃, halo, aryl, substituted aryl, heteroaryl, substituted heteroaryl or a heterocyclic group, where R is independently alkyl, alkenyl, alkynyl, alkylaryl, aryl or cycloalkyl.

7. The photo-responsive compound according to claim 1, wherein
the photochromic unit is at least one selected from the group of a diarylethene, a spiropyran, a spirooxazine, and a rhodamine.

8. An organic memory device comprising in sequence, an anode, an active layer comprising a cathode and a photo-responsive coordination compound having formula (I): 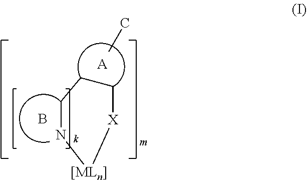
wherein
a) X is oxygen, sulphur, selenium, NR or PR where R is an alkyl, alkylaryl, cycloalkyl, alkoxy, benzyl, aryl, substituted aryl, heteroaryl, substituted heteroaryl or a heterocyclic group;
b) A is cyclic structure derivative of substituted or unsubstituted arene or heteroarene;
c) B, which is optionally fused with or singly-bonded to A, is cyclic structure derivative of substituted or unsubstituted heterocyclic group containing one or more nitrogen atoms;
d) C is a photochromic unit;
e) [MLₙ] represents the coordination unit containing a metal or main group element M and L is a ligand;
f) k is a number of rings in the cyclic structure derivatives and k is an integer from 0 to 2;
g) n is a number of ligands and n is an integer from 0 to 4; and
h) m is a number of the photochromic ligand and m is an integer from 1 to 4.

9. The organic memory device according to claim 8 in which the photo-responsive coordination compound is thermally evaporated or spin-coated.

10. The organic memory device according to claim 8 in which the photo-responsive coordination compound is a material for an active layer in the fabrication of the organic memory device with photo-controllable performance.

11. The organic memory device according to claim 8, wherein
the photochromic unit is at least one selected from the group of a diarylethene, a spiropyran, a spirooxazine, and a rhodamine.

12. A photo-responsive electron-transporting material comprising a coordination compound having a formula (I): 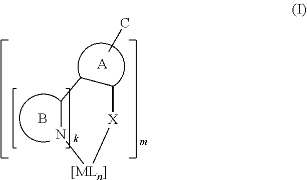
wherein
a) X is oxygen, sulphur, selenium, NR or PR where R is an alkyl, alkylaryl, cycloalkyl, alkoxy, benzyl, aryl, substituted aryl, heteroaryl, substituted heteroaryl or a heterocyclic group;
b) A is cyclic structure derivative of substituted or unsubstituted arene or heteroarene;
c) B, which is optionally fused with or singly-bonded to A, is cyclic structure derivative of substituted or unsubstituted heterocyclic group containing one or more nitrogen atoms;
d) C is a photochromic unit;
e) [MLₙ] represents the coordination unit containing a metal or main group element M and L is a ligand;
f) k is a number of rings in the cyclic structure derivatives and k is an integer from 0 to 2;
g) n is a number of ligands and n is an integer from 0 to 4; and
h) m is a number of the photochromic ligand and m is an integer from 1 to 4.

13. The photo-responsive electron-transporting material according to claim 12, wherein ring A is cyclic structure derivative where the cyclic structure is independently selected from a 5- or 6-membered arene or heteroarene, wherein the arene is one or more of benzene, naphthalene, anthracene, pyrene, fluorene and derivatives thereof, and the heteroarene is one or more of pyridine, pyrazole, imidazole, oxazole, isoxazole, thiazole, isothiazole, isoquinoline, pyrrole, pyrazine, pyridazine, pyrimidine, benzimidazole, benzothiazole, indole, triazole, tetrazole, pyran, oxadiazole, triazine, tetrazine, and derivatives thereof.

14. The photo-responsive electron-transporting material according to claim 12, wherein ring B is cyclic structure derivative where the cyclic structure is independently selected from a 5- or 6-membered nitrogen-containing heteroarene or heterocycle, wherein the heteroarene or heterocycle is one or more of pyridine, pyrazole, imidazole, oxazole, isoxazole, thiazole, isothiazole, isoquioline, pyrrole, pyrazine, pyridazine, pyrimidine, benzimidazole, benzothiazole, indole, triazole, tetrazole, pyran, oxadiazole, triazine, tetrazine, and derivatives thereof.

15. The photo-responsive electron-transporting material according to claim 12, wherein the photochromic unit is at least one selected from the group of a diarylethene, a spiropyran, a spirooxazine, and a rhodamine.
